(19) United States
(12) Patent Application Publication
(10) Pub. No.: US 2022/0277134 A1
(43) Pub. Date: Sep. 1, 2022

(54) DOCUMENT CREATION SUPPORT APPARATUS, METHOD, AND PROGRAM

(71) Applicant: FUJIFILM Corporation, Tokyo (JP)

(72) Inventor: Keigo NAKAMURA, Tokyo (JP)

(73) Assignee: FUJIFILM Corporation, Tokyo (JP)

(21) Appl. No.: 17/746,978

(22) Filed: May 18, 2022

Related U.S. Application Data
(63) Continuation of application No. PCT/JP2020/044367, filed on Nov. 27, 2020.

(30) Foreign Application Priority Data
Nov. 29, 2019 (JP) 2019-216046

(51) Int. Cl. G06F 40/166; G06F 40/274

(52) U.S. Cl. CPC G06F 40/166; G06F 40/274

(57) ABSTRACT

A document creation support apparatus includes at least one processor, and the processor is configured to display a sentence including at least one content on a display, specify a correction content to be added to the sentence or deleted from the sentence based on input or designation of at least some characters of the correction content, and correct the sentence based on the correction content.

### CROSS REFERENCE TO RELATED APPLICATIONS

The present application is a Continuation of PCT International Application No. PCT/JP2020/044367, filed on Nov. 27, 2020, which claims priority to Japanese Patent Application No. 2019-216046, filed on Nov. 29, 2019. Each application above is hereby expressly incorporated by reference, in its entirety, into the present application.

### BACKGROUND

### Technical Field

The present disclosure relates to a document creation support apparatus, a method, and a program that support creation of documents such as medical documents.

### Related Art

In recent years, advances in medical devices, such as computed tomography (CT) apparatuses and magnetic resonance imaging (MRI) apparatuses, have enabled image diagnosis using high-resolution medical images with higher quality. In particular, since a region of a lesion can be accurately specified by image diagnosis using CT images, MM images, and the like, appropriate treatment is being performed based on the specified result.

In addition, image diagnosis is also made by analyzing a medical image by computer-aided diagnosis (CAD) using a learning model in which machine learning is performed by deep learning or the like, discriminating properties such as the shape, density, position, and size of structures of interest such as abnormal shadow candidates included in the medical images, and acquiring them as an analysis result. The analysis result acquired by CAD is associated with examination information such as a patient name, gender, age, and a modality that has acquired the medical image, and is saved in a database. The medical image and the analysis result are transmitted to a terminal of a radiologist who interprets the medical images. The radiologist interprets the medical image by referring to the transmitted medical image and analysis result and creates an interpretation report, in his or her own terminal.

Meanwhile, with the improvement of the performance of the CT apparatus and the MRI apparatus described above, the number of medical images to be interpreted is also increasing. However, since the number of radiologists has not kept up with the number of medical images, it is desired to reduce the burden of the image interpretation work of the radiologists. Therefore, various methods have been proposed to support the creation of medical documents such as interpretation reports. For example, JP2019-153250A proposes various methods for generating a sentence to be described in an interpretation report based on keywords input by a radiologist and information indicating a property of a structure of interest (hereinafter referred to as property information) included in an analysis result of a medical image (see JP2019-153250A). In the methods described in JP2019-153250A, a sentence for medical care (hereinafter referred to as a medical sentence) is created by using a learning model in which machine learning is performed, such as a recurrent neural network trained to generate a sentence from characters representing the input property information. By automatically generating the medical sentence as in the method described in JP2019-153250A, it is possible to reduce a burden on a radiologist at the time of creating a medical document such as an interpretation report.

On the other hand, although various methods for automatically generating a sentence have been proposed as described above, correction by the radiologist is often required. Therefore, a method for easily correcting the generated sentence is desired.

For example, JP2002-117026A proposes a method in which a text document includes one or more text components, an incorrect text component is identified from the one or more text components, a list of alternatives is displayed based on a partial input of a desired alternative for the incorrect text component, and the incorrect text component in the text document is replaced with a selected alternative. By using the method described in JP2002-117026A, it is possible to correct an incorrect part in a sentence without imposing a burden on a user.

However, in the method described in JP2002-117026A, the user needs to perform an operation of selecting an alternative to be corrected from the alternative list. Further, in a case where an alternative of the desired content is not present in the alternative list, the method described in JP2002-117026A cannot efficiently correct the sentence.

### SUMMARY OF THE INVENTION

The present disclosure has been made in consideration of the above circumstances, and an object thereof is to efficiently correct a sentence.

According to an aspect of the present disclosure, there is provided a document creation support apparatus comprising at least one processor, in which the processor is configured to display a sentence including at least one content on a display, specify a correction content to be added to the sentence or deleted from the sentence based on input or designation of at least some characters of the correction content, and correct the sentence based on the correction content.

In the document creation support apparatus according to the aspect of the present disclosure, the processor may be configured to further analyze an image to derive property information indicating a property of a structure of interest included in the image as the content.

In the document creation support apparatus according to the aspect of the present disclosure, the processor may be configured to generate a sentence related to the image based on the property information.

In the document creation support apparatus according to the aspect of the present disclosure, the processor may be configured to specify the correction content based on the property information.

In the document creation support apparatus according to the aspect of the present disclosure, the processor may be configured to specify the content included in the sentence, and change the specified content based on the correction content.

In the document creation support apparatus according to the aspect of the present disclosure, the processor may be configured to correct the sentence according to a style of the sentence before correction.

In the document creation support apparatus according to the aspect of the present disclosure, the processor may be configured to generate a plurality of correction candidates, and correct the sentence based on a correction candidate according to a style of the sentence before correction among the plurality of correction candidates.

In the document creation support apparatus according to the aspect of the present disclosure, the processor may be configured to generate a plurality of correction candidates, display the plurality of correction candidates on the display, receive selection of a correction candidate to be used for the sentence from the plurality of displayed correction candidates, and correct the sentence based on the selected correction candidate.

In the document creation support apparatus according to the aspect of the present disclosure, the processor may be configured to the processor is configured to correct the sentence such that the correction content and a content included in the sentence before correction match.

According to another aspect of the present disclosure, there is provided a document creation support method comprising: displaying a sentence including at least one content on a display; specifying a correction content to be added to the sentence or deleted from the sentence based on input or designation of at least some characters of the correction content; and correcting the sentence based on the correction content.

In addition, a program for causing a computer to execute the document creation support method according to the aspect of the present disclosure may be provided.

According to the aspects of the present disclosure, it is possible to efficiently correct a sentence.

### BRIEF DESCRIPTION OF THE DRAWINGS

FIG. 1 is a diagram showing a schematic configuration of a medical information system to which a document creation support apparatus according to a first embodiment of the present disclosure is applied.

FIG. 2 is a diagram showing a schematic configuration of the document creation support apparatus according to the first embodiment.

FIG. 3 is a diagram showing an example of teacher data for training a first learning model.

FIG. 4 is a diagram schematically showing a configuration of a recurrent neural network.

FIG. 5 is a diagram showing an example of teacher data for training a third learning model.

FIG. 6 is a diagram showing an example of a display screen of a medical sentence according to the first embodiment.

FIG. 7 is a diagram showing an example of a display screen of the medical sentence in which correction contents are being input.

FIG. 8 is a diagram showing an example of a table showing a derivation result of property information.

FIG. 9 is a diagram schematically showing a configuration of a recurrent neural network in a sentence correction unit.

FIG. 10 is a diagram showing an example of teacher data for training a fourth learning model.

FIG. 11 is a diagram showing an example of a display screen of a medical sentence according to the first embodiment.

FIG. 12 is a diagram showing another example of a medical sentence according to the first embodiment.

FIG. 13 is a flowchart showing a process performed in the first embodiment.

FIG. 14 is a diagram showing an example of a display screen of a medical sentence displaying a plurality of correction candidates according to a second embodiment.

FIG. 15 is a flowchart showing a process performed in the second embodiment.

FIG. 16 is a diagram showing an example of a property table.

### DETAILED DESCRIPTION

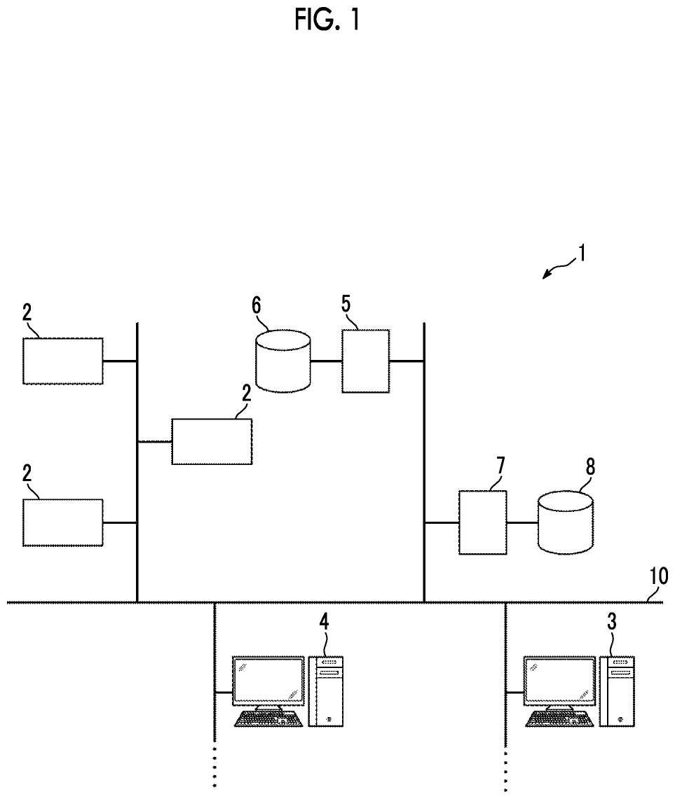

Hereinafter, embodiments of the present disclosure will be described with reference to the drawings. FIG. 1 is a diagram showing a schematic configuration of a medical information system to which a document creation support apparatus according to a first embodiment of the present disclosure is applied. A medical information system 1 shown in FIG. 1 is, based on an examination order from a doctor in a medical department using a known ordering system, a system for imaging an examination target part of a subject, storing a medical image acquired by the imaging, interpreting the medical image by a radiologist and creating an interpretation report, and viewing the interpretation report and observing the medical image to be interpreted in detail by the doctor in the medical department that is a request source. As shown in FIG. 1, the medical information system 1 is configured to include a plurality of modalities (imaging apparatuses) 2, a plurality of interpretation workstations (WS) 3 that are interpretation terminals, a medical department workstation (WS) 4, an image server 5, an image database 6, an interpretation report server 7, and an interpretation report database 8 that are communicably connected to each other through a wired or wireless network 10.

Each apparatus is a computer on which an application program for causing each apparatus to function as a component of the medical information system 1 is installed. The application program is stored in a storage apparatus of a server computer connected to the network 10 or in a network storage in a state in which it can be accessed from the outside, and is downloaded to and installed on the computer in response to a request. Alternatively, the optimization support program is recorded on a recording medium, such as a digital versatile disc (DVD) or a compact disc read only memory (CD-ROM), and distributed, and is installed on the computer from the recording medium.

The modality 2 is an apparatus that generates a medical image showing a diagnosis target part of the subject by imaging the diagnosis target part. Specifically, examples of the modality include a simple X-ray imaging apparatus, a CT apparatus, an MRI apparatus, a positron emission tomography (PET) apparatus, and the like. A medical image generated by the modality 2 is transmitted to the image server 5 and is saved therein.

The interpretation WS 3 encompasses the document creation support apparatus according to the present embodiment. The configuration of the interpretation WS 3 will be described later.

The medical department WS 4 is a computer used by a doctor in a medical department to observe an image in detail, view an interpretation report, create an electronic medical record, and the like, and is configured to include a processing apparatus, a display, and an input device such as a keyboard and a mouse. In the medical department WS 4, each process such as creating a medical record (electronic medical record) of a patient, requesting the image server 5 to view an image, displaying an image received from the image server 5, automatically detecting or highlighting a lesion-like portion in the image, requesting the interpretation report server 7 to view an interpretation report, and displaying the interpretation report received from the interpretation report server 7 is performed by executing a software program for each process.

The image server 5 is a general-purpose computer on which a software program that provides a function of a database management system (DBMS) is installed. The image server 5 comprises a storage in which the image database 6 is configured. This storage may be a hard disk apparatus connected to the image server 5 by a data bus, or may be a disk apparatus connected to a storage area network (SAN) or a network attached storage (NAS) connected to the network 10. In a case where the image server 5 receives a request to register a medical image from the modality 2, the image server 5 prepares the medical image in a format for a database and registers the medical image in the image database 6.

Image data of the medical image acquired by the modality 2 and accessory information are registered in the image database 6. The accessory information includes, for example, an image identification (ID) for identifying each medical image, a patient ID for identifying a subject, an examination ID for identifying an examination, a unique ID (UID: unique identification) allocated for each medical image, examination date and examination time at which a medical image is generated, the type of modality used in an examination for acquiring a medical image, patient information such as the name, age, and gender of a patient, an examination part (imaging part), imaging information (an imaging protocol, an imaging sequence, an imaging method, imaging conditions, the use of a contrast medium, and the like), and information such as a series number or a collection number when a plurality of medical images are acquired in one examination.

In addition, in a case where a viewing request from the interpretation WS 3 is received through the network 10, the image server 5 searches for a medical image registered in the image database 6 and transmits the searched for medical image to the interpretation WS 3 that is a request source.

The interpretation report server 7 incorporates a software program for providing a function of a database management system to a general-purpose computer. In a case where the interpretation report server 7 receives a request to register an interpretation report from the interpretation WS 3, the interpretation report server 7 prepares the interpretation report in a format for a database and registers the interpretation report in the interpretation report database 8. Further, in a case where the request to search for the interpretation report is received, the interpretation report is searched from the interpretation report database 8.

In the interpretation report database 8, for example, an interpretation report is registered in which information, such as an image ID for identifying a medical image to be interpreted, a radiologist ID for identifying an image diagnostician who performed the interpretation, a lesion name, position information of a lesion, findings, and confidence of the findings, is recorded.

In the present embodiment, it is assumed that the medical image is a three-dimensional CT image consisting of a plurality of tomographic images with the lung as a diagnosis target, and an interpretation report on an abnormal shadow included in the lung is created as a medical document by interpreting the CT image. The medical image is not limited to the CT image, and any medical image such as an MRI image and a simple two-dimensional image acquired by a simple X-ray imaging apparatus can be used.

The network 10 is a wired or wireless network that connects various apparatuses in a hospital to each other. In a case where the interpretation WS 3 is installed in another hospital or clinic, the network 10 may be configured to connect local area networks of respective hospitals through the Internet or a dedicated line.

Hereinafter, the interpretation WS 3 according to the present embodiment will be described in detail. The interpretation WS 3 is a computer used by a radiologist of a medical image to interpret the medical image and create an interpretation report, and is configured to include a processing apparatus, a display, and an input device such as a keyboard and a mouse. In the interpretation WS 3, each process such as requesting the image server 5 to view a medical image, various kinds of image processing on the medical image received from the image server 5, displaying the medical image, an analysis process on the medical image, highlighting the medical image based on the analysis result, creating the interpretation report based on the analysis result, supporting the creation of an interpretation report, requesting the interpretation report server 7 to register and view the interpretation report, and displaying the interpretation report received from the interpretation report server 7 is performed by executing a software program for each process. Note that, in these processes, processes other than those performed by the document creation support apparatus according to the present embodiment are performed by a well-known software program, and therefore the detailed description thereof will be omitted here. In addition, processes other than the processes performed by the document creation support apparatus according to the present embodiment may not be performed in the interpretation WS 3, and a computer that performs the processes may be separately connected to the network 10, and in response to a processing request from the interpretation WS 3, the requested process may be performed by the computer.

The interpretation WS 3 encompasses the document creation support apparatus according to the first embodiment. Therefore, a document creation support program according to the present embodiment is installed on the interpretation WS 3. The document creation support program is stored in the storage apparatus of the server computer connected to the network or in the network storage in a state in which it can be accessed from the outside, and is downloaded to and installed on the interpretation WS 3 in response to a request. Alternatively, the medical document creation program is recorded on a recording medium such as a DVD or a CD-ROM, distributed, and is installed on the interpretation WS 3 from the recording medium.

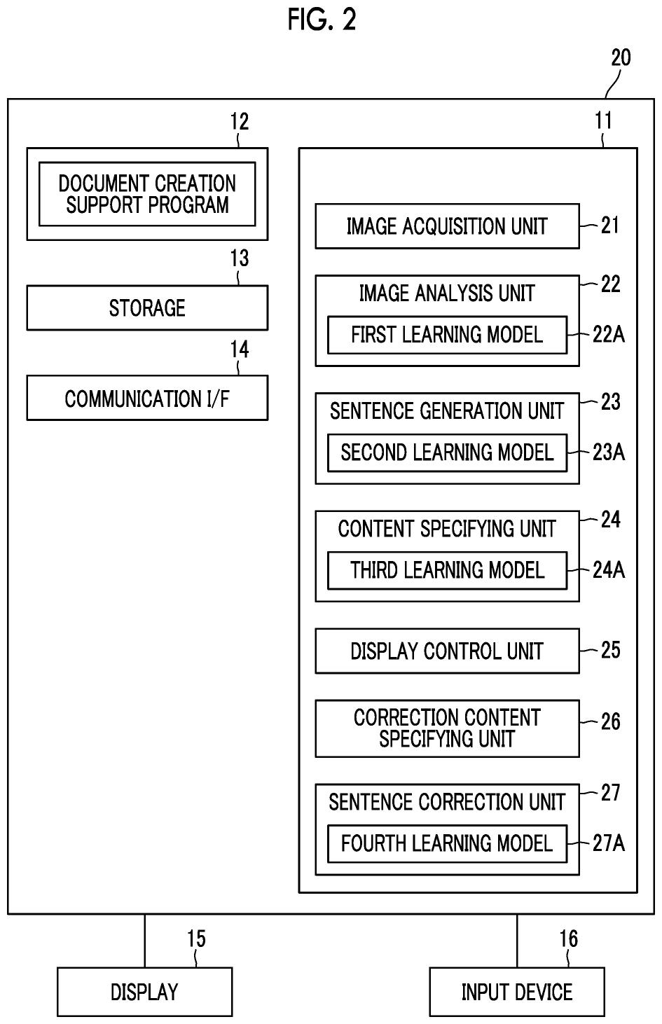

FIG. 2 is a diagram showing a schematic configuration of the document creation support apparatus according to the first embodiment, which is realized by installing the document creation support program on the interpretation WS 3. As shown in FIG. 2, a document creation support apparatus 20 comprises a central processing unit (CPU) 11, a memory 12, a storage 13, and a communication interface (I/F) 14 as a standard computer configuration. Further, a display 15 such as a liquid crystal display and an input device 16 such as a keyboard and a mouse are connected to the document creation support apparatus 20. The CPU 11 corresponds to a processor.

The storage 13 consists of a storage device, such as a hard disk or a solid state drive (SSD). The storage 13 stores various kinds of information including medical images and information necessary for processing of the document creation support apparatus 20, which are acquired from the image server 5 through the network 10.

The communication I/F 14 is a network interface that controls transmission of various information between an external apparatus and the document creation support apparatus 20 via the network 10.

Further, the memory 12 stores a document creation support program. As processes to be executed by the CPU 11, the document creation support program defines an image acquisition process of acquiring a medical image, an image analysis process of deriving property information indicating a property of the structure of interest included in the medical image by analyzing the medical image, a sentence generation process of generating a medical sentence related to the medical image based on the property information, a content specifying process of specifying the content representing the property related to the structure of interest included in the medical sentence by analyzing the medical sentence, a display control process of displaying the generated medical sentence on the display 15, a correction content specifying process of specifying a correction content based on the input or designation of at least some characters of the correction content to be added to the medical sentence or deleted from the medical sentence, and a sentence correction process of correcting the medical sentence based on the correction content.

Then, the CPU 11 executes the processes according to the document creation support program, so that the computer functions as an image acquisition unit 21, an image analysis unit 22, a sentence generation unit 23, a content specifying unit 24, a display control unit 25, and a correction content specifying unit 26 and a sentence correction unit 27.

The image acquisition unit 21 consists of an interface connected to the network 10, and acquires a medical image for creating an interpretation report from the image server 5 according to an instruction from the input device 16 by the radiologist who is an operator.

The image analysis unit 22 analyzes the medical image to derive property information indicating the property of the structure of interest such as an abnormal shadow candidate included in the medical image. For this purpose, the image analysis unit 22 has a first learning model 22A in which machine learning is performed so as to discriminate an abnormal shadow candidate in the medical image and discriminate the property of the discriminated abnormal shadow candidate. In the present embodiment, the first learning model 22A consists of a convolutional neural network (CNN) in which deep learning is performed using teacher data so as to discriminate whether or not each pixel (voxel) in the medical image represents an abnormal shadow candidate, and discriminate a property of a pixel in a case where the pixel represents an abnormal shadow candidate.

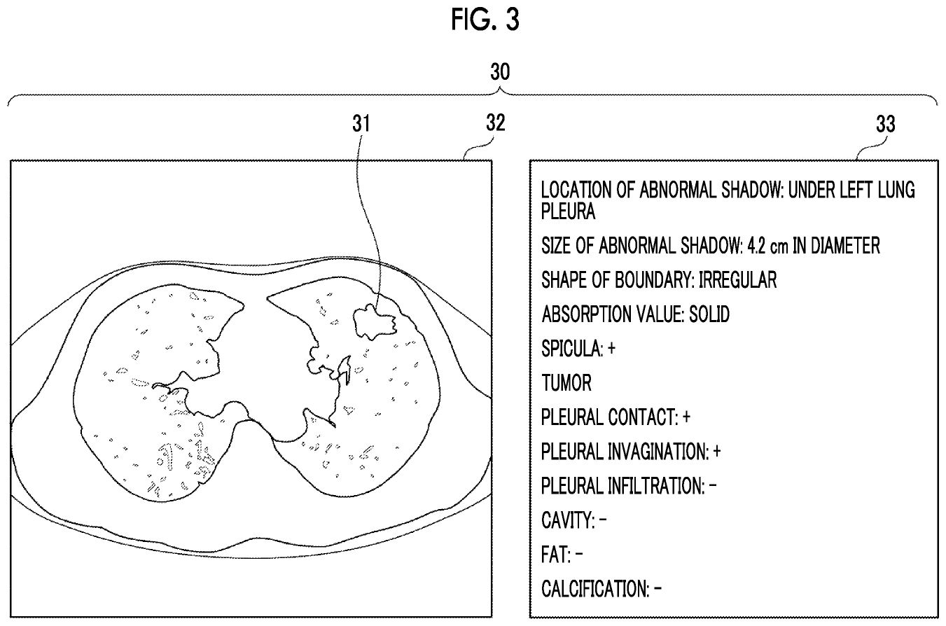

FIG. 3 is a diagram showing an example of teacher data for training a first learning model. As shown in FIG. 3, teacher data 30 includes a medical image 32 including an abnormal shadow 31 and property information 33 about the abnormal shadow. In the present embodiment, it is assumed that the abnormal shadow 31 is a lung nodule, and the property information 33 indicates a plurality of properties of the lung nodule. For example, as the property information 33, the location of the abnormal shadow, the size of the abnormal shadow, the shape of the boundary (clear and irregular), the type of absorption value (solid and frosted glass type), the presence or absence of spicula, whether it is a tumor or a nodule, the presence or absence of pleural contact, the presence or absence of the pleural invagination, the presence or absence of pleural infiltration, the presence or absence of a cavity, the presence or absence of fat, and the presence or absence of calcification are used. Regarding the abnormal shadow 31 included in the teacher data 30 shown in FIG. 3, the property information 33 indicates, as shown in FIG. 3, that the location of the abnormal shadow is under the left lung pleura, the size of the abnormal shadow is 4.2 cm in diameter, the shape of the boundary is irregular, the absorption value is a solid type, spicula is present, it is a tumor, pleural contact is present, pleural invagination is present, pleural infiltration is absent, a cavity is absent, fat is absent, and calcification is absent. In addition, in FIG. 3, + is given in the case of “present”, and − is given in the case of “absent”. The first learning model 22A is constructed by learning a neural network using a large amount of teacher data as shown in FIG. 3. For example, by using the teacher data 30 shown in FIG. 3, the first learning model 22A is trained to discriminate the abnormal shadow 31 included in the medical image 32 in a case where the medical image 32 shown in FIG. 3 is input, and output the property information 33 shown in FIG. 3 with respect to the abnormal shadow 31.

The property information derived by the image analysis unit 22 is saved in the storage 13 as a table showing a derivation result. The table showing a derivation result will be described later.

Further, as the first learning model 22A, any learning model such as, for example, a support vector machine (SVM) can be used in addition to the convolutional neural network.

Note that the learning model for detecting the abnormal shadow candidate from the medical image and the learning model for detecting the property information of the abnormal shadow candidate may be constructed separately.

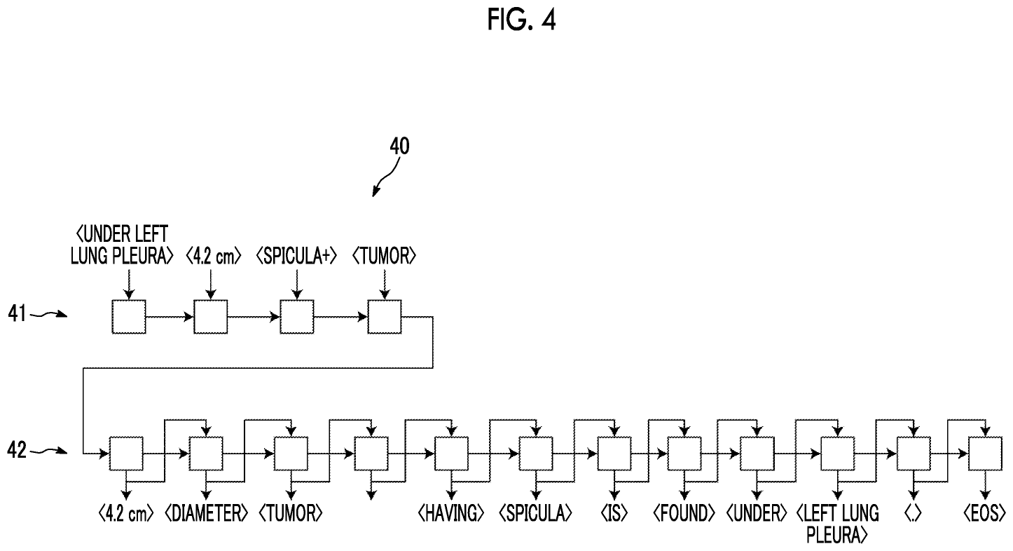

The sentence generation unit 23 generates a medical sentence by using the property information derived by the image analysis unit 22. The sentence generation unit 23 consists of a second learning model 23A that has been trained to generate a sentence from the input information. As the second learning model 23A, for example, a recurrent neural network can be used. FIG. 4 is a diagram schematically showing a configuration of a recurrent neural network in the sentence generation unit 23. As shown in FIG. 4, the recurrent neural network 40 consists of an encoder 41 and a decoder 42. The property information derived by the image analysis unit 22 is input to the encoder 41. For example, property information indicating “under left lung pleura”, “4.2 cm”, “spicula +” and “tumor” is input to the encoder 41. The decoder 42 is trained to document character information, and generates a sentence from the input property information. Specifically, from the above-mentioned property information indicating “under left lung pleura”, “4.2 cm”, “spicula +” and “tumor”, a medical sentence “A 4.2 cm diameter tumor having spicula is found under the left lung pleura.” is generated. In FIG. 4, “EOS” indicates the end of the sentence (End Of Sentence).

In this way, in order to output the medical sentence by inputting the property information, the recurrent neural network 40 is constructed by learning the encoder 41 and the decoder 42 using a large amount of teacher data consisting of a combination of the property information and the medical sentence.

The content specifying unit 24 specifies a term representing a property included in the medical sentence generated by the sentence generation unit 23 as the content of the medical sentence. For this purpose, the content specifying unit 24 has a third learning model 24A in which machine learning is performed so as to specify a term representing a property included in a medical sentence. In the present embodiment, the third learning model 24A consists of a convolutional neural network in which deep learning is performed using the teacher data so as to discriminate the terms representing the properties included in the input medical sentence in a case where the medical sentence is input.

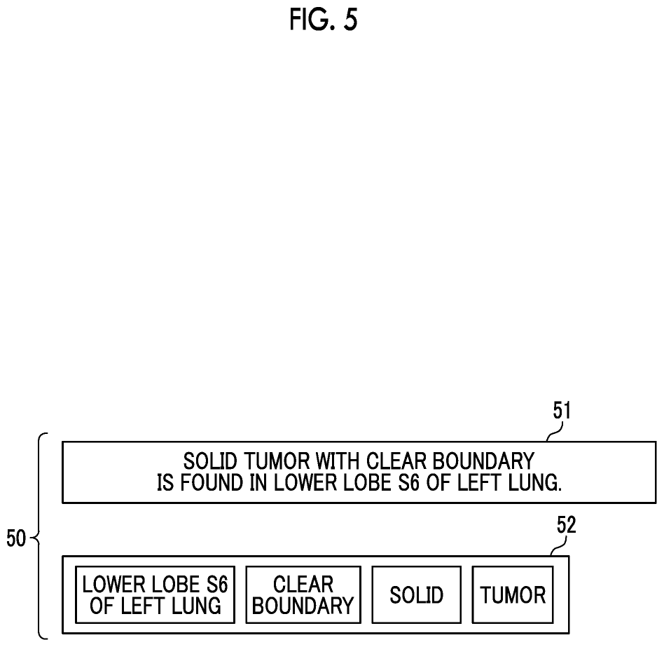

FIG. 5 is a diagram showing an example of teacher data for training the third learning model. As shown in FIG. 5, the teacher data 50 includes a medical sentence 51 and terms 52 representing the properties included in the medical sentence 51. The medical sentence 51 shown in FIG. 5 is “A solid tumor with a clear boundary is found in the lower lobe S6 of the left lung”, and the terms 52 representing the properties are “lower lobe S6 of the left lung”, “clear boundary”, “solid”, and “tumor” included in the medical sentence 51. The third learning model 24A is constructed by learning a neural network using a large amount of teacher data as shown in FIG. 5. For example, by using the teacher data 50 shown in FIG. 5, the third learning model 24A is trained such that the terms 52 shown in FIG. 5 are output as the contents included in the sentence in a case where the medical sentence 51 shown in FIG. 5 is input. A specified content is associated with the medical image and is saved in the storage 13.

Further, as the third learning model 24A, for example, any learning model such as a support vector machine and a recurrent neural network can be used, in addition to the convolutional neural network.

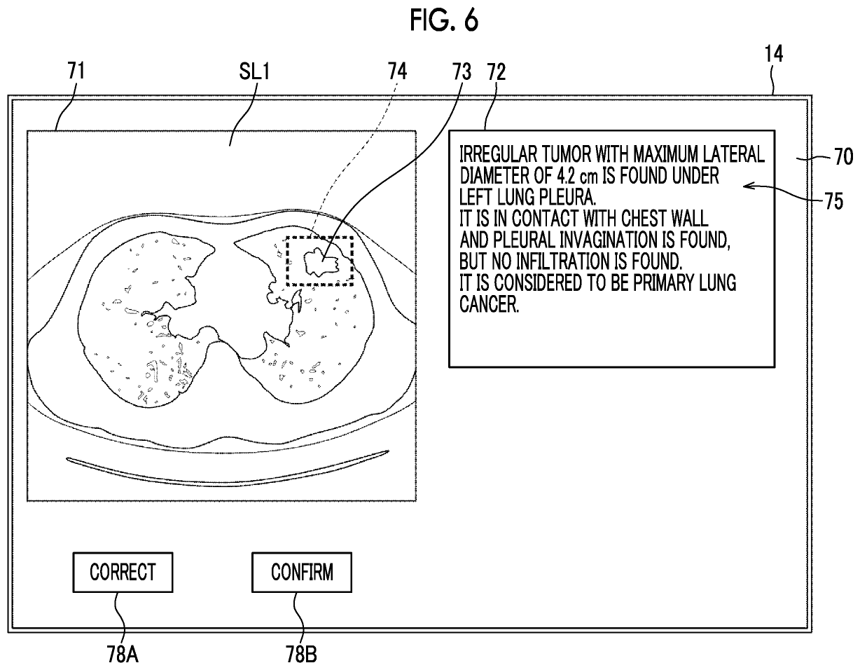

The display control unit 25 displays the medical sentence generated by the sentence generation unit 23 on the display 15. FIG. 6 is a diagram showing an example of a display screen of a medical sentence according to the first embodiment. As shown in FIG. 6, the display screen 70 includes an image display region 71 and a sentence display region 72. In the image display region 71, a slice image SL1 that is most likely to specify the abnormal shadow candidate detected by the image analysis unit 22 is displayed. The slice image SL1 includes an abnormal shadow candidate 73, and the abnormal shadow candidate 73 is surrounded by a rectangular region 74.

In the sentence display region 72, medical sentences 75 generated by the sentence generation unit 23 are displayed. The medical sentences 75 are “An irregular tumor with a maximum lateral diameter of 4.2 cm is found under the left lung pleura. It is in contact with the chest wall and pleural invagination is found, but no infiltration is found. It is considered to be primary lung cancer.”. In the medical sentence 75, the contents specified by the content specifying unit 24 are “under left lung pleura”, “irregular”, “4.2 cm”, “tumor”, “contact with the chest wall”, “with pleural invagination”, “no infiltration”, and “primary lung cancer”.

Below the image display region 71, a correction button 78A and a confirmation button 78B are displayed.

The radiologist interprets the abnormal shadow candidate 73 in the slice image SL1 displayed in the image display region 71, and determines the suitability of the medical sentence 75 displayed in the sentence display region 72.

In a case where the radiologist wants to correct the medical sentence 75, he or she uses the input device 16 to select the correction button 78A. Thereby, the medical sentence 75 displayed in the sentence display region 72 can be manually corrected by input from the input device 16. Further, by selecting the confirmation button 78B, the medical sentence 75 displayed in the sentence display region 72 can be confirmed with its contents. In this case, the medical sentence 75 is transcribed in an interpretation report, and the interpretation report to which the medical sentence 75 has been transcribed is transmitted to the interpretation report server 7 together with the slice image SL1 and stored therein.

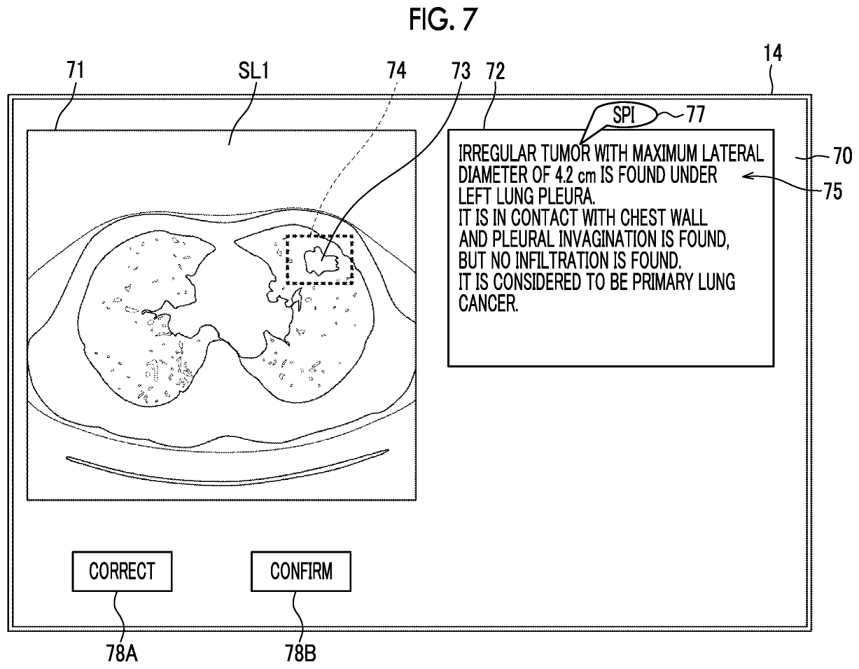

At the time when the radiologist selects the correction button 78A to correct the medical sentence 75, in a case where there is a property that is included in the abnormal shadow 31, but is lacking in the medical sentence 75, the radiologist makes the correction to add the lacking property. In this case, the radiologist inputs the lacking property using the input device 16. For example, in the present embodiment, it is assumed that spicula is found in the abnormal shadow 31, but the medical sentence 75 lacks the description of spicula. In this case, the radiologist inputs the characters “spicula” using the input device 16. At that time, as shown in FIG. 7, first, the characters “s” and “pi” are input. In FIG. 7, for the sake of description, the input characters are displayed in a pop-up 77, but the method of inputting the characters is not limited thereto. Characters may be input at the cursor position in the sentence.

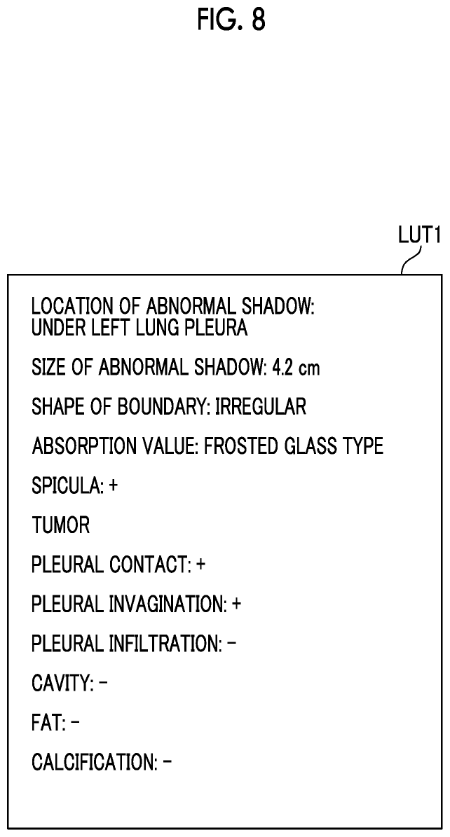

The correction content specifying unit 26 specifies a correction content to be added to the medical sentence or deleted from the medical sentence based on input or designation of at least some characters of the correction content. Further, in the present embodiment, the correction content is specified by referring to a table showing a derivation result of the property information by the image analysis unit 22, which is saved in the storage 13. FIG. 8 is a diagram showing an example of the table showing a derivation result. As shown in FIG. 8, a table LUT1 representing the derivation result includes a plurality of pieces of property information such as the location of an abnormal shadow, the size of the abnormal shadow, the shape of the boundary (clear and irregular), the type of absorption value (solid and frosted glass type), the presence or absence of spicula, whether it is a tumor or a nodule, the presence or absence of pleural contact, the presence or absence of the pleural invagination, the presence or absence of pleural infiltration, the presence or absence of a cavity, the presence or absence of fat, and the presence or absence of calcification. In the table LUT1 shown in FIG. 8, the location of the abnormal shadow is under the left lung pleura, the size of the abnormal shadow is 4.2 cm in diameter, the shape of the boundary is irregular, the absorption value is a frosted glass type, spicula is present, it is a tumor, pleural contact is present, pleural invagination is present, pleural infiltration is absent, a cavity is absent, fat is absent, and calcification is absent. In addition, also in FIG. 8, + is given in the case of “present”, and − is given in the case of “absent”.

The correction content specifying unit 26 specifies the correction content by referring to the table LUT1 saved in the storage 13. In the present embodiment, the correction content specifying unit 26 recognizes the characters “s” and “pi” input by the radiologist, and specifies the correction content as “spicula” including the characters “s” and “pi” by further referring to the table LUT1. In addition, in a case where the input character is “tu”, the correction content specifying unit 26 specifies the correction content as “tumor”, and in a case where the input characters are “ir” and “re”, the correction content specifying unit 26 specifies the correction content as “irregular”.

Meanwhile, in a case of deleting a part of the medical sentence 75 displayed in the sentence display region 72, the radiologist specifies property information included in the medical sentence 75 by using the input device 16. For example, in a case of deleting the sentence “pleural invagination is found, but”, the radiologist “designates the characters of the property information in the order of “pleu”, “ral”, “invagin”, and so on by using the input device 16 to move the cursor in the displayed medical sentence 75. The designation of characters also includes selecting a character by moving the cursor in order from the first character. The correction content specifying unit 26 specifies the correction content as “pleural invagination” by referring to the table LUT1 by the characters “pleu”, “ral”, and “invagin” designated by the radiologist. The correction content specifying unit 26 specifies the correction content as “cavity” in a case where the designated character is “ca”, and specifies the correction content as “calcification” in a case where the designated character is “calc”.

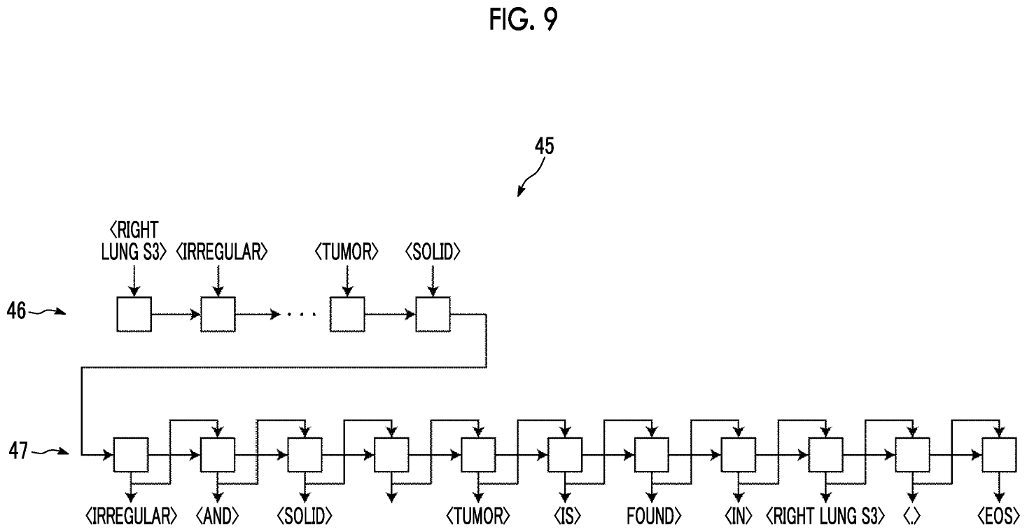

The sentence correction unit 27 corrects the medical sentence 75 based on the correction content specified by the correction content specifying unit 26. For this purpose, the sentence correction unit 27 has a fourth learning model 27A in which machine learning is performed so as to output a corrected medical sentence (hereinafter referred to as a correction medical sentence) in a case where the medical sentence and the correction content are input. As the fourth learning model 27A, for example, a recurrent neural network can be used. FIG. 9 is a diagram schematically showing a configuration of a recurrent neural network in the sentence correction unit 27. As shown in FIG. 9, a recurrent neural network 45 consists of an encoder 46 and a decoder 47.

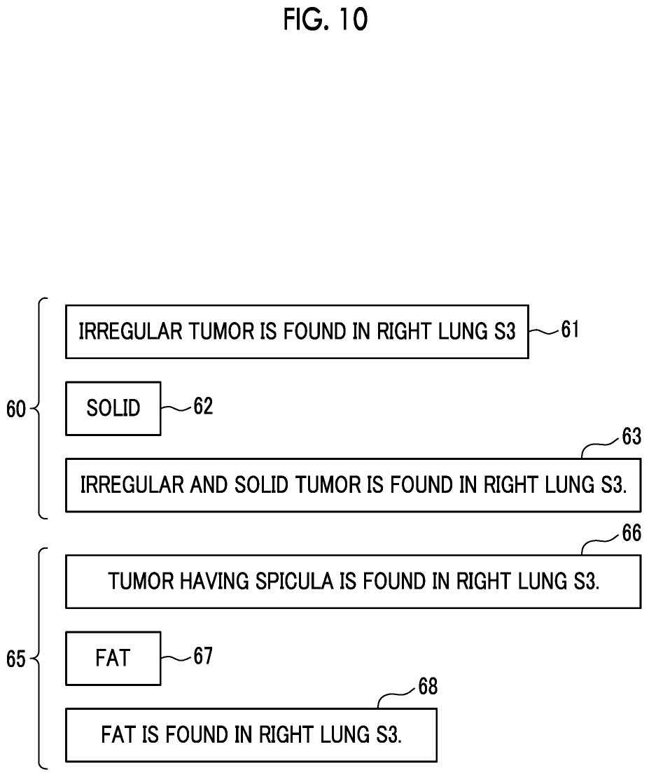

FIG. 10 is a diagram showing an example of teacher data for training the fourth learning model 27A. Note that FIG. 10 shows two types of teacher data. As shown in FIG. 10, teacher data 60 includes a medical sentence 61, a correction content 62, and a correction medical sentence 63. The medical sentence 61 is “An irregular tumor is found in the right lung S3.”, the correction content 62 is “solid”, and the correction medical sentence 63 is “An irregular and solid tumor is found in the right lung S3.”. The teacher data 60 is obtained by adding a wording including the correction content 62 to the medical sentence 61 to generate the correction medical sentence 63.

On the other hand, teacher data 65 includes a medical sentence 66, a correction content 67, and a correction medical sentence 68. The medical sentence 66 is “A tumor having spicula is found in right lung S3.”, the correction content 67 is “fat”, and the correction medical sentence 68 is “Fat is found in the right lung S3.”. The teacher data 65 is obtained by adding the correction content 67 indicating benign to the medical sentence 66 in which the description indicating malignancy is added, and deleting the description indicating malignancy to generate the correction medical sentence 68.

At the time of learning, as shown in FIG. 9, the property information included in the medical sentence 61 of the teacher data 60 is input to the encoder 46 of the recurrent neural network 45, and the correction content 62 (“solid” in FIG. 9) is input thereto. Then, the encoder 46 and the decoder 47 of the recurrent neural network 45 are learned so that the decoder 47 outputs the correction medical sentence 63. Further, in a case where learning is performed using the teacher data 65, as in the case of using the teacher data 60, the property information included in the medical sentence 66 of the teacher data 65 is input to the encoder 46 of the recurrent neural network 45, and the correction content 67 is input thereto. Then, the encoder 46 and the decoder 47 of the recurrent neural network 45 are learned so that the decoder 47 outputs the correction medical sentence 68.

The fourth learning model 27A is constructed by learning the recurrent neural network 45 using a large amount of teacher data as shown in FIG. 10. For example, by using the teacher data 60 shown in FIG. 10, the fourth learning model 27A is trained such that the correction medical sentence 63 shown in FIG. 10 is output in a case where the medical sentence 61 and the correction content 62 shown in FIG. 10 are input.

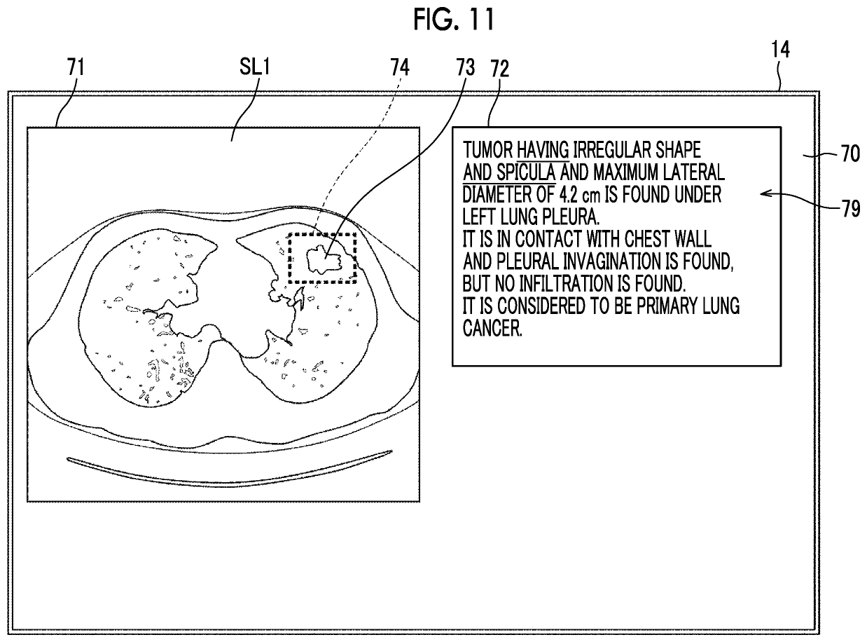

As a result, as shown in FIG. 7, in a case where the characters “s” and “pi” are input and the correction content is specified as “spicula”, the sentence correction unit 27 corrects the medical sentence 75 and correction medical sentences of “A tumor having an irregular shape and spicula and a maximum lateral diameter of 4.2 cm is found under the left lung pleura. It is in contact with the chest wall and pleural invagination is found, but no infiltration is found. It is considered to be primary lung cancer.” are generated. As shown in FIG. 11, the display control unit 25 displays a correction medical sentence 79 in the sentence display region 72. In FIG. 11, the corrected portion in the correction medical sentence 79 is underlined.

At this time, the sentence correction unit 27 corrects the medical sentence 75 according to the style of the medical sentence 75. Specifically, in a case where the medical sentence 75 is a formal tone, the medical sentence 75 is corrected so that the correction medical sentence 79 is also a formal tone. Further, in a case where the medical sentence 75 is an informal tone, the medical sentence 75 is corrected so that the correction medical sentence 79 is also an informal tone.

Further, the sentence correction unit 27 may generate a plurality of correction candidates based on the correction content, select a correction candidate according to the style of the medical sentence 75 before correction from among the plurality of generated correction candidates, and correct the medical sentence 75. For example, in a case where the correction content is “spicula” for the above-mentioned sentence “An irregular tumor with a maximum lateral diameter of 4.2 cm is found under the left lung pleura.”, in addition to “A tumor having an irregular shape and spicula and a maximum lateral diameter of 4.2 cm is found under the left lung pleura.”, a plurality of correction candidates such as “A tumor having an irregular shape and spicula and a maximum lateral diameter of 4.2 cm found under the left lung pleura.” and “An irregular tumor with spicula and a maximum lateral diameter of 4.2 cm found under the left lung pleura.” are generated. Then, the sentence correction unit 27 selects one correction candidate from among the plurality of generated correction candidates according to the style of the medical sentence 75 before the correction, and generates the correction medical sentence 79. In this case, among the above-mentioned three correction candidates, “A tumor having an irregular shape and spicula and a maximum lateral diameter of 4.2 cm is found under the left lung pleura.”, which is a formal tone, is selected, and the correction medical sentence 79 is generated.

The plurality of correction candidates can be generated, for example, by applying a beam search method described in “https://geekyisawesome.blogspot.com/2016/10/using-beam-search-to-generate-most.html” to the recurrent neural network 45 constituting the fourth learning model 27A. The beam search method is a method of searching for a word that appears next to a certain word in consideration of the probability of appearance of the word that appears next to a certain word. In another embodiment, the sentence correction unit 27 applies the beam search method to the recurrent neural network 45 to generate correction candidates for a plurality of medical sentences having a high probability of appearance of the word.

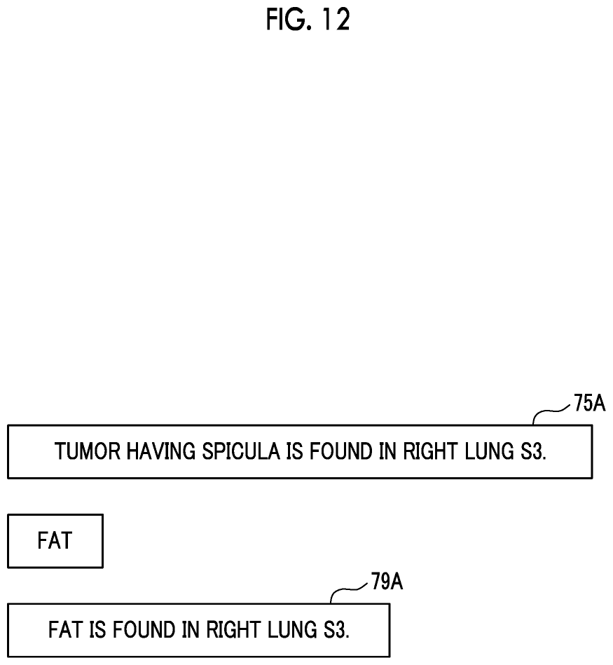

As described above, the fourth learning model 27A of the sentence correction unit 27 is trained using the teacher data 65 shown in FIG. 10. Therefore, for example, as shown in FIG. 12, it is assumed that the medical sentence 75A of “A tumor having a spicula is found in the right lung S3” is displayed in the sentence display region 72, and the correction content is “fat”. In this case, since spicula is a content indicating malignancy and fat is a content indicating benign, the medical sentence 75A is corrected to delete the wording related to malignancy included in the medical sentence 75A, and the correction medical sentence 79A of “Fat is found in the right lung S3.” is displayed.

On the other hand, in a case where the content included in the medical sentence 75 is designated, the sentence correction unit 27 deletes the wording including the designated content from the medical sentence. For example, in the medical sentence 75 shown in FIG. 6, in a case where “pleu”, “ral”, and “invagin” are designated, the correction content is specified as “pleural invagination”. Then, the sentence correction unit 27 deletes the wording “pleural infiltration is found, but”. As a result, the correction medical sentence 79 becomes “An irregular tumor with a maximum lateral diameter of 4.2 cm is found under the left lung pleura. It is in contact with the chest wall and no infiltration is found. It is considered to be primary lung cancer.”. In this case, “under left lung pleura”, “irregular”, “4.2 cm”, “tumor”, “contact with the chest wall”, “no infiltration”, and “primary lung cancer”, excluding the content related to pleural invagination among the contents included in the medical sentence 75, are input to the recurrent neural network 45 of the fourth learning model 27A of the sentence correction unit 27, and the correction medical sentence 79 is generated.

Further, in the present embodiment, the content included in the correction medical sentence 79 is different from the content included in the medical sentence 75 before the correction according to the correction of the medical sentence by the sentence correction unit 27. For example, in a case where the medical sentence 75 is “An irregular tumor with a maximum lateral diameter of 4.2 cm is found under the left lung pleura. It is in contact with the chest wall and pleural invagination is found, but no infiltration is found. It is considered to be primary lung cancer.”, the contents specified by the content specifying unit 24 are “under left lung pleura”, “irregular”, “4.2 cm”, “tumor”, “contact with the chest wall”, “with pleural invagination”, “no infiltration”, and “primary lung cancer”.

On the other hand, in a case where the correction medical sentence 79 is “A tumor having an irregular shape and spicula and a maximum lateral diameter of 4.2 cm is found under the left lung pleura. It is in contact with the chest wall and pleural invagination is found, but no infiltration is found. It is considered to be primary lung cancer.”, the correction medical sentence 79 includes “spicula” as content, which is not included in the medical sentence 75 before the correction. Therefore, the sentence correction unit 27 changes the content by adding “spicula” to the content of the medical sentence 75 saved in the storage 13. Thereby, the contents of medical sentences saved in the storage 13 become “under left lung pleura”, “irregular”, “spicula”, “4.2 cm”, “tumor”, “contact with the chest wall”, “with pleural invagination”, “no infiltration, and “primary lung cancer”.

The content of the medical sentence 75 and the content of the correction medical sentence 79 may be included in the display screen 70 and displayed.

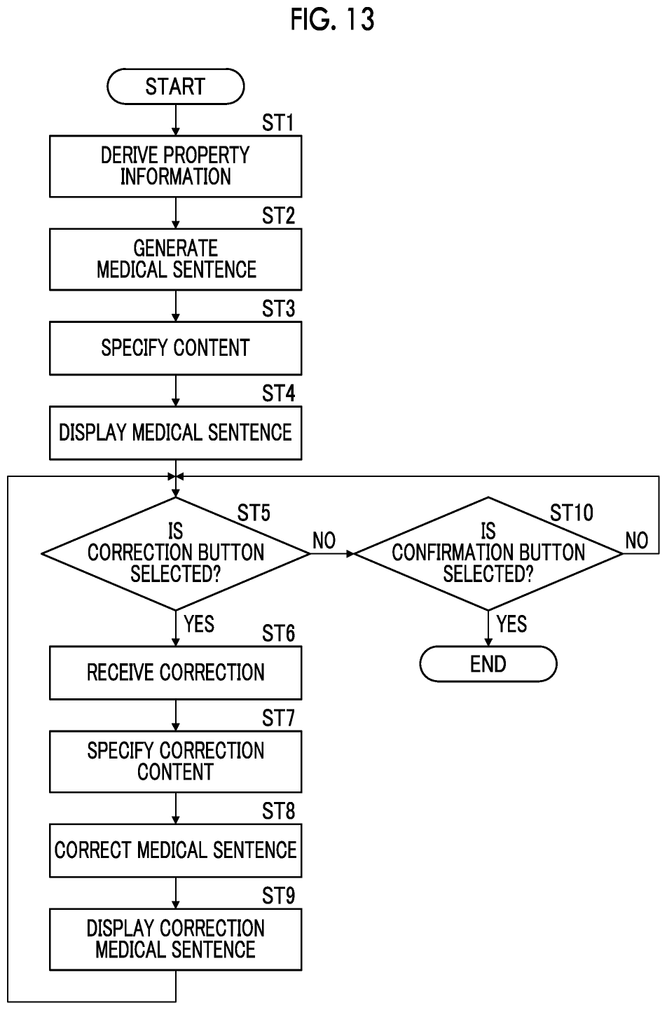

Next, a process performed in the first embodiment will be described. FIG. 13 is a flowchart showing a process performed in the present embodiment. It is assumed that the medical image to be interpreted is acquired from the image server 5 by the image acquisition unit 21 and is saved in the storage 13. The process is started in a case where an instruction to create an interpretation report is given by the radiologist, and the image analysis unit 22 analyzes the medical image to derive property information indicating the property of the structure of interest such as an abnormal shadow candidate included in the medical image (Step ST1). Next, the sentence generation unit 23 generates a medical sentence related to the medical image based on the property information (Step ST2). Subsequently, the content specifying unit 24 analyzes the medical sentence generated by the sentence generation unit 23 to specify the term representing the property related to the structure of interest included in the medical sentence as the content (Step ST3). Then, the display control unit 25 displays the medical sentence generated by the sentence generation unit 23 on the sentence display region 72 of the display screen 70 displayed on the display 15 (Step ST4).

Next, the display control unit 25 determines whether or not the correction button 78A displayed on the display screen is selected (Step ST5). In a case where Step ST5 is affirmative, the display control unit 25 receives the correction of the medical sentence displayed in the sentence display region 72 using the input device 16 (Step ST6). In a case where the correction using the input device 16 is started, the correction content specifying unit 26 specifies a correction content to be added to the medical sentence or deleted from the medical sentence based on input or designation of at least some characters of the correction content (Step ST7). Then, the sentence correction unit 27 corrects the medical sentence based on the correction content (Step ST8). Thereby, a correction medical sentence is generated. Next, the display control unit 25 displays the correction medical sentence in the sentence display region 72 of the display screen 70 (Step ST9), and returns to the process of Step ST5.

In a case where Step ST5 is negative, the display control unit 25 determines whether or not the confirmation button 78B is selected (Step ST10). In a case where Step ST10 is negative, the process returns to Step ST5. In a case where Step ST10 is affirmative, the display control unit 25 transcribes the medical sentence to the interpretation report, and transmits the interpretation report to which the medical sentence is transcribed to the interpretation report server 7 together with the slice image SL1 (interpretation report transmission: Step ST11), and the process ends.

In this way, in the first embodiment, the medical sentence including at least one content is displayed on the display 15. Then, a correction content to be added to the medical sentence or deleted from the sentence is specified based on input or designation of at least some characters of the correction content. In addition, the sentence is corrected based on the correction content. Therefore, the medical sentence can be corrected according to the correction intention of the radiologist who is the user. In addition, since the correction content is specified based on the input or designation of at least some of the characters of the correction content, the burden on the radiologist who makes the correction can also be reduced. Therefore, according to the present embodiment, the medical sentence can be efficiently corrected.

Next, a second embodiment of the present disclosure will be described. Since the configuration of a document creation support apparatus according to the second embodiment is the same as that of the document creation support apparatus 20 according to the first embodiment shown in FIG. 2 and only the processing to be performed is different, detailed description of the apparatus will be omitted here. The document creation support apparatus according to the second embodiment is different from that of the first embodiment in that the sentence correction unit 27 generates a plurality of correction candidates, the display control unit 25 displays the plurality of correction candidates, and the sentence correction unit 27 generates a correction medical sentence based on the correction candidate selected from the plurality of correction candidates.

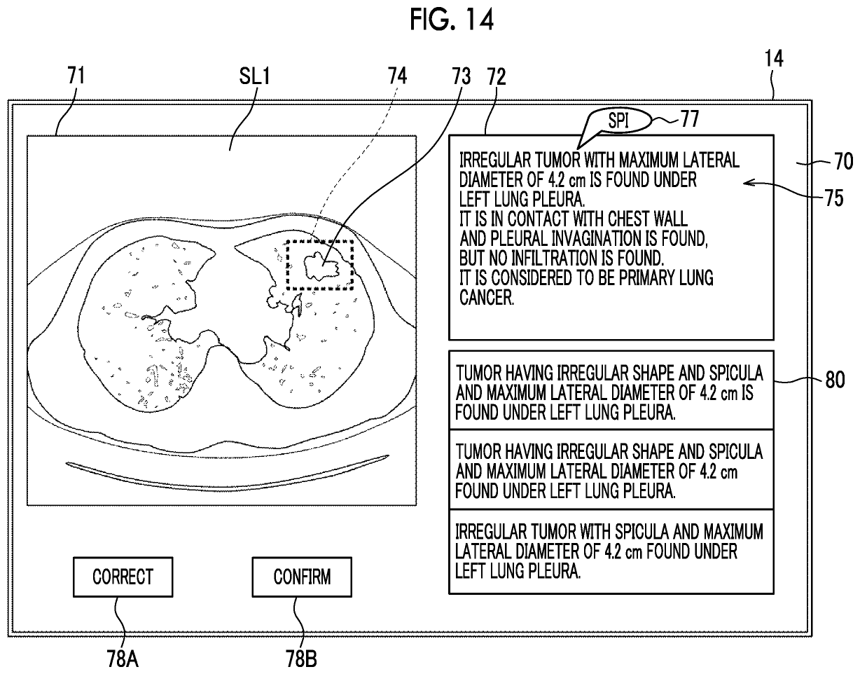

FIG. 14 is a diagram showing a display screen of a medical sentence displaying a plurality of correction candidates in the second embodiment. Note that FIG. 14 shows a state in which the characters “s” and “pi” are input, as in FIG. 7. As shown in FIG. 14, in a case where the radiologist inputs the characters “s” and “pi” using the input device 16, the correction content specifying unit 26 specifies the correction content as “spicula” by referring to the LUT1, and the sentence correction unit 27 generates a plurality of correction candidates based on the correction content. Then, the display control unit 25 displays a plurality of correction candidate display regions 80 below the sentence display region 72. Then, for example, in the correction candidate display regions 80, three correction candidates: “A tumor having an irregular shape and spicula and a maximum lateral diameter of 4.2 cm is found under the left lung pleura.”, “A tumor having an irregular shape and spicula and a maximum lateral diameter of 4.2 cm found under the left lung pleura.”, and “An irregular tumor with spicula and a maximum lateral diameter of 4.2 cm found under the left lung pleura.” are displayed. The number of correction candidates is not limited to three.

The radiologist selects a desired correction candidate from the displayed three correction candidates using the input device 16. As a result, the correction medical sentence 79 is generated based on the selected correction candidates. For example, in a case where the radiologist selects the top correction candidate from the displayed four correction candidates, the correction medical sentences of “A tumor having an irregular shape and spicula and a maximum lateral diameter of 4.2 cm is found under the left lung pleura. It is in contact with the chest wall and pleural invagination is found, but no infiltration is found. It is considered to be primary lung cancer.” are generated as in the first embodiment above.

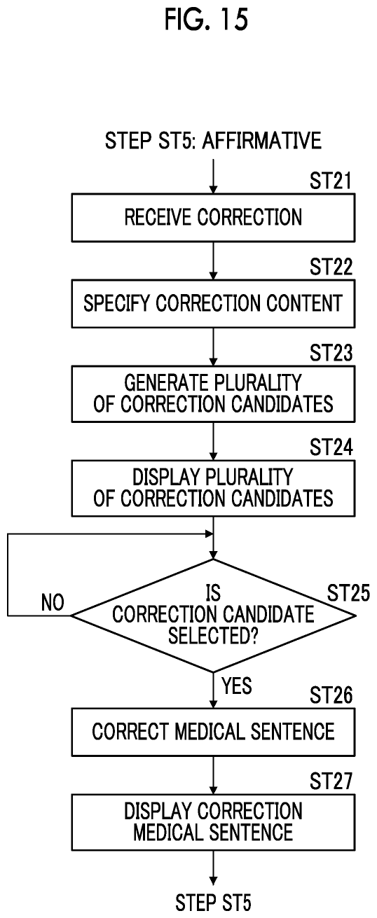

Next, a process performed in the second embodiment will be described. FIG. 15 is a flowchart showing a process performed in the second embodiment. The process performed in the second embodiment is different from the process performed in the first embodiment shown in FIG. 13 only in the process after Step ST5 is affirmative. Therefore, in FIG. 15, only the process after the affirmation of Step ST5 in FIG. 13 will be described.

In a case where Step ST5 in FIG. 13 is affirmative, the display control unit 25 receives the correction of the medical sentence displayed in the sentence display region 72 using the input device 16 (Step ST21). In a case where the correction using the input device 16 is started, the correction content specifying unit 26 specifies a correction content to be added to the medical sentence or deleted from the medical sentence based on input or designation of at least some characters of the correction content (Step ST22). Then, the sentence correction unit 27 generates a plurality of correction candidates for the medical sentence based on the correction content (Step ST23). Further, the display control unit 25 displays the plurality of correction candidates in the correction candidate display regions 80 (Step ST24).

Then, in a case where any one of the plurality of correction candidates is selected (Step ST25: affirmative), the sentence correction unit 27 corrects the medical sentence based on the selected correction candidate (Step ST26). Thereby, a correction medical sentence is generated. Next, the display control unit 25 displays the correction medical sentence in the sentence display region 72 of the display screen 70 (Step ST27), and returns to the process of Step ST5.

In this way, in the second embodiment, a plurality of correction candidates are generated, the selection of one correction candidate desired by the radiologist is received from the plurality of correction candidates, and the medical sentence is corrected based on the selected correction candidate. Therefore, the correction medical sentence can be generated so as to include the correction candidate desired by the radiologist, and as a result, the burden on the radiologist who creates the interpretation report can be reduced.

In each of the above embodiments, the medical image is analyzed and the medical sentence is generated in the interpretation WS 3, but the present disclosure is not limited thereto. For example, the medical information system 1 in the present embodiment may be provided with an analysis server that analyzes a medical image and generates a medical sentence, and the analysis server may analyze the medical image and generate the medical sentence. In this case, the generated medical sentence is transmitted from the analysis server to the interpretation WS 3 together with a table showing a derivation result of property information, and the medical sentence is displayed and corrected in the interpretation WS 3 in the same manner as in the above embodiments.

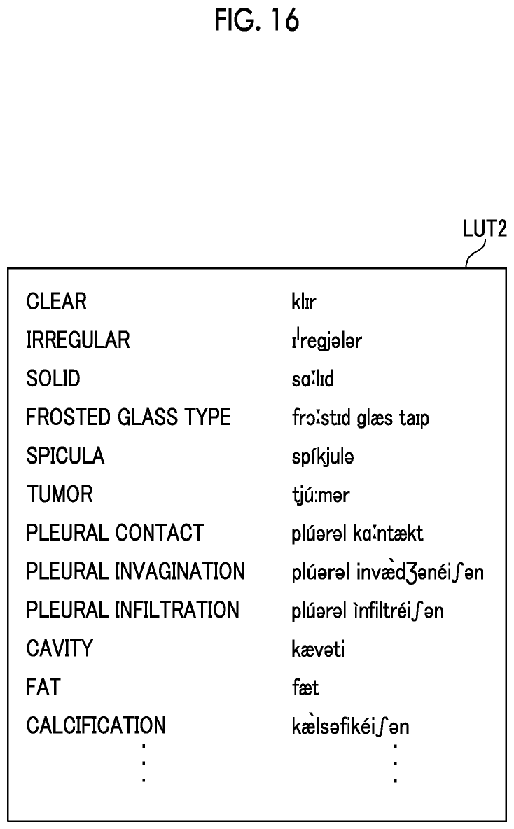

Further, in each of the above embodiments, the correction content specifying unit 26 specifies the correction content by referring to the table LUT1 showing the derivation result shown in FIG. 8, but the present disclosure is not limited thereto. For example, as shown in FIG. 16, a property table LUT2 in which a plurality of pieces of property information that can be detected in a lung nodule is associated with a phonetic spelling representing the property information may be saved in the storage 13, and the correction content may be specified by referring to the property table LUT2. Here, in the property table LUT2, various property information such as clear, irregular, solid, frosted glass type, spicula, and tumor is associated with a phonetic spelling representing each property information.

In this case, the correction content specifying unit 26 specifies the correction content by referring to the property table LUT2 saved in the storage 13. For example, the correction content specifying unit 26 specifies the correction content as a “spicula” including the characters “s” and “pi” by the characters “s” and “pi” input by the radiologist. In addition, in a case where the input character is “tju”, the correction content specifying unit 26 specifies the correction content as “tumor”, and in a case where the input characters are “i” and “re”, the correction content specifying unit 26 specifies the correction content as “irregular”.

Further, in the above embodiments, the process of the present disclosure is applied to the sentence generated by the sentence generation unit 23 based on the property information derived by the image analysis unit 22, but the present disclosure is not limited thereto. The technique of the present disclosure can also be applied to medical sentences created by the radiologist by inputting by himself/herself. In this case, the property information derived by the image analysis unit 22 is not present, and thus the correction content specifying unit 26 specifies the correction content by referring to the property table LUT2 as shown in FIG. 16.

Further, in each of the above embodiments, the creation support process for the medical document such as the interpretation report is performed by generating the medical sentence using the medical image with the lung as the diagnosis target, but the diagnosis target is not limited to the lung. In addition to the lung, any part of a human body such as a heart, liver, brain, and limbs can be diagnosed. In this case, for each learning model of the image analysis unit 22, the sentence generation unit 23, the content specifying unit 24, and the sentence correction unit 27, learning models that perform the analysis process, the sentence generation process, the content specifying process, and the sentence correction process according to the diagnosis target are prepared, a learning model that performs the analysis process, the sentence generation process, the content specifying process, and the sentence correction process according to the diagnosis target is selected, and a process of generating a medical sentence is executed.

In addition, in each of the above embodiments, although the technique of the present disclosure is applied to the case of creating an interpretation report as a medical document, the technique of the present disclosure can also be applied to a case of creating medical documents other than the interpretation report, such as an electronic medical record and a diagnosis report.

Further, in each of the above embodiments, the medical sentence is generated using the medical image, but the present disclosure is not limited thereto. Of course, the technique of the present disclosure can also be applied even in a case where a sentence targeting any image other than a medical image is generated.

Further, in each of the above embodiments, for example, as hardware structures of processing units that execute various kinds of processing, such as the image acquisition unit 21, the image analysis unit 22, the sentence generation unit 23, the content specifying unit 24, the display control unit 25, the correction content specifying unit 26, and the sentence correction unit 27, various processors shown below can be used. As described above, the various processors include a programmable logic device (PLD) as a processor of which the circuit configuration can be changed after manufacture, such as a field programmable gate array (FPGA), a dedicated electrical circuit as a processor having a dedicated circuit configuration for executing specific processing such as an application specific integrated circuit (ASIC), and the like, in addition to the CPU as a general-purpose processor that functions as various processing units by executing software (programs).

One processing unit may be configured by one of the various processors, or may be configured by a combination of the same or different kinds of two or more processors (for example, a combination of a plurality of FPGAs or a combination of the CPU and the FPGA). In addition, a plurality of processing units may be configured by one processor.

As an example where a plurality of processing units are configured by one processor, first, there is a form in which one processor is configured by a combination of one or more CPUs and software as typified by a computer, such as a client or a server, and this processor functions as a plurality of processing units. Second, there is a form in which a processor for realizing the function of the entire system including a plurality of processing units via one integrated circuit (IC) chip as typified by a system on chip (SoC) or the like is used. In this way, various processing units are configured by using one or more of the above-described various processors as hardware structures.

Furthermore, as the hardware structure of the various processors, more specifically, an electrical circuit (circuitry) in which circuit elements such as semiconductor elements are combined can be used.

What is claimed is:

1. A document creation support apparatus comprising at least one processor,
wherein the processor is configured to
display a sentence including at least one content on a display,
specify a correction content to be added to the sentence or deleted from the sentence based on input or designation of at least some characters of the correction content, and
correct the sentence based on the correction content.

2. The document creation support apparatus according to claim 1, wherein the processor is configured to further analyze an image to derive property information indicating a property of a structure of interest included in the image as the content.

3. The document creation support apparatus according to claim 2, wherein the processor is configured to generate a sentence related to the image based on the property information.

4. The document creation support apparatus according to claim 2, wherein the processor is configured to specify the correction content based on the property information.

5. The document creation support apparatus according to claim 1, wherein the processor is configured to
specify the content included in the sentence, and
change the specified content based on the correction content.

6. The document creation support apparatus according to claim 1, wherein the processor is configured to correct the sentence according to a style of the sentence before correction.

7. The document creation support apparatus according to claim 1, wherein the processor is configured to
generate a plurality of correction candidates, and
correct the sentence based on a correction candidate according to a style of the sentence before correction among the plurality of correction candidates.

8. The document creation support apparatus according to claim 1, wherein the processor is configured to
generate a plurality of correction candidates,
display the plurality of correction candidates on the display,
receive selection of a correction candidate to be used for the sentence from the plurality of displayed correction candidates, and
correct the sentence based on the selected correction candidate.

9. The document creation support apparatus according to claim 1, wherein the processor is configured to correct the sentence such that the correction content and a content included in the sentence before correction match.

10. A document creation support method comprising:
displaying a sentence including at least one content on a display;
specifying a correction content to be added to the sentence or deleted from the sentence based on input or designation of at least some characters of the correction content; and
correcting the sentence based on the correction content.

11. A non-transitory computer-readable storage medium that stores a document creation support program causing a computer to execute procedures comprising:
displaying a sentence including at least one content on a display;
specifying a correction content to be added to the sentence or deleted from the sentence based on input or designation of at least some characters of the correction content; and
correcting the sentence based on the correction content.
